(19) United States
(12) Patent Application Publication
(10) Pub. No.: US 2010/0291622 A1
(43) Pub. Date: Nov. 18, 2010

(54) NOVEL COMPOUND, PROCESS FOR PRODUCTION THEREOF, AND USE THEREOF

(75) Inventors: Hiroshi Tomoda, Kanagawa (JP); Junji Inokoshi, Kanagawa (JP); Rokuro Masuma, Kanagawa (JP); Satoshi Omura, Kanagawa (JP)

Correspondence Address: WPAT, PC;INTELLECTUAL PROPERTY ATTORNEYS, 2030 MAIN STREET, SUITE 1300, IRVINE, CA, 92614, US

(73) Assignee: SCHOOL JURIDICAL PERSON KITASATO INSTITUTE, Tokyo (JP)

(21) Appl. No.: 12/681,202

(22) PCT Filed: Sep. 12, 2008
(86) PCT No.: PCT/JP2008/066924

§ 371 Date: Jul. 26, 2010

(30) Foreign Application Priority Data
Sep. 14, 2007 (JP) 2007-239762
Sep. 12, 2008 (JP) PCT/JP2008/066924

(51) Int. Cl. C12P 29/00; C07C 237/26; C07C 49/727; C12N 1/20

(52) U.S. Cl. 435/64; 552/203; 552/202; 435/252.1

(57) ABSTRACT

The present invention provides compounds which exhibit an inhibitory effect on undecaprenyl diphosphate synthase of microbes and thereby they are expected to be used clinically as pharmaceutical agents for infectious diseases caused by bacteria.

The process comprises culturing a microbe belonging to Penicillium and having ability of producing an FKI-3368 substance in or on a nutrient medium to accumulate the FKI-3368 substance in the nutrient medium and collecting the FKI-3368 substance from the culture.

### CROSS-REFERENCE TO RELATED APPLICATIONS

The present application claims the benefit of priority of an International Patent Application PCT/JP2008/066924 filed Sep. 12, 2008 which in turn claims benefit of priority of a Japanese Patent Application No. 2007-239762, filed Sep. 14, 2007, both with the Japan Patent Office, the contents of which are incorporated herein by reference in their entirety.

### FIELD OF THE INVENTION

The present invention relates to novel compounds (referred to as FKI-3368 substances in the present application.) and the production process and the use thereof, more specifically, the present invention relates to compounds mentioned above having isoprenoid metabolism inhibitory activity and the production process and the use thereof.

### BACKGROUND OF THE INVENTION

Various beta-lactam antibiotics, aminoglycosides, macrolides, glycopeptides, quinolones, etc. have been conventionally used for the prevention and treatment of bacterial infection. Recently, however, bacteria showing tolerance to these antibiotics have been increasing, and antibiotics which are different from the conventional types of antibiotics are demanded.

Undecaprenyl diphosphate synthase is an enzyme generating undecaprenyl diphosphate using farnesyl 2-phosphate and isoprenyl 2-phosphate as substrates. This is an enzyme protein which, participating in the synthesis of cell walls, etc., is essential to the life support of bacteria and accordingly it is conceived that discovery of an agent inhibiting this enzyme can contribute to solving the problem mentioned above and such an agent is expected to be used clinically as a new therapeutic agent for intractable infectious diseases due to multiple drug resistant bacteria such as MRSA or VRE. However, although there has been proposed a gene product derived from Streptococcus genus as a target in developing antibiotics relating to the undecaprenyl diphosphate synthase (Japanese Patent Application Laid-Open No. 2002-527049 (WO00/21544)), no antibacterial agents inhibiting this enzyme have been put in practical use yet.

### SUMMARY OF THE INVENTION

An object of the present invention is to provide a novel compound which has an inhibitory effect on undecaprenyl diphosphate synthase of microbes and thereby can be clinically used as an antibacterial agent, and to provide a production process thereof.

### Means for Solving the Problems

The present inventors have conducted a series of extended studies on metabolites produced by microbes, and consequently have found that a substance having an undecaprenyl diphosphate synthetase inhibitory activity is produced in the culture of FKI-3368 strain newly separated from the soil. Subsequently, the inventors separated and purified active substances which inhibit the undecaprenyl diphosphate synthase from the said culture and found substances having the chemical structure represented by the general formula (I) and thus completed the present invention.

The compounds of the present invention exhibit an inhibitory effect on undecaprenyl diphosphate synthase of microbes and thereby they are expected to be used clinically as pharmaceutical agents for intractable infectious diseases caused by bacteria.

### BRIEF DESCRIPTION OF THE DRAWINGS

FIG. 1 shows the ultraviolet absorption spectrum (in C₂H₅OH) of the FKI-3368-1 substance of the present invention.

FIG. 2 shows the infrared absorption spectrum (KBr method) of the FKI-3368-1 substance of the present invention.

FIG. 3 shows the proton nuclear magnetic resonance spectrum (CDCl₃) of the FKI-3368-1 substance of the present invention.

FIG. 4 shows the carbon nuclear magnetic resonance spectrum (CDCl₃) of the FKI-3368-1 substance of the present invention.

FIG. 5 shows the ultraviolet absorption spectrum (in C₂H₅OH) of the FKI-3368-2 substance of the present invention.

FIG. 6 shows the infrared absorption spectrum (KBr method) of the FKI-3368-2 substance of the present invention.

FIG. 7 shows the proton nuclear magnetic resonance spectrum (CDCl₃) of the FKI-3368-2 substance of the present invention.

FIG. 8 shows the carbon nuclear magnetic resonance spectrum (CDCl₃) of the FKI-3368-2 substance of the present invention.

### DETAILED DESCRIPTION OF THE INVENTION

### A. FKI-3368 Substances

The novel compounds (FKI-3368 substances) of the present invention have the following structure.

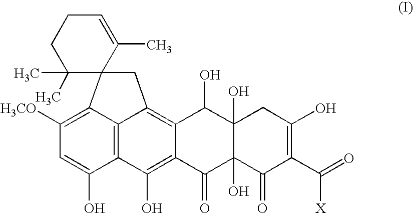

In the formula, X is a hydroxyl group, an alkyl group having 1 to 3 carbon atoms which may be substituted or an amino group which may be substituted, and the alkyl group having 1 to 3 carbon atoms includes a methyl group, an ethyl group, a propyl group and an isopropyl group. The substituent group is not limited in particular, but, for example, a hydroxyl group, halogen atoms, an alkoxy group and an amino group are included. In addition, the present invention includes pharmaceutically acceptable salts or derivatives thereof.

Here, the salts include anyone of combinations of an anionic residue and a cation or combinations of a cationic residue and an anion, and the examples of the cation include alkali metals, alkaline-earth metals and any other metal cations and nitrogen-containing cations such as ammonium ion, and the examples of the anion include halogens, sulfate ion and nitrate ion. The derivatives may include esters and reaction products with a protecting group which is commonly used pharmaceutically.

Examples of these compounds include the following compounds:

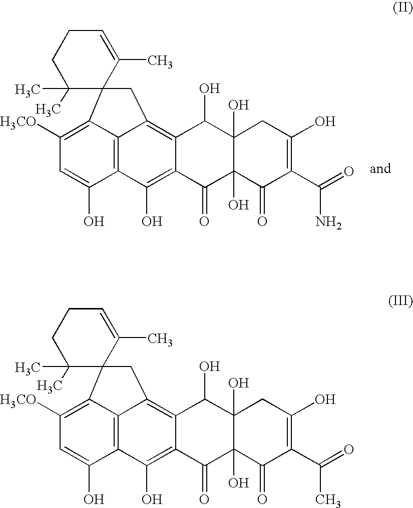

(Hereinbelow, these are referred to as FKI-3368-1 substance and FKI-3368-2 substance in the present application.).

These compounds are novel and have an antibiotic activity, more specifically an inhibitory activity on undecaprenyl diphosphate synthase.

### B. Production Process of FKI-3368 Substances

The present invention also provides a production process of an FKI-3368 substance which comprises culturing a microbe (hereinbelow, referred to as “FKI-3368 substance producing microbe”) belonging to genus Penicillium and having an ability of producing an FKI-3368 substance in or on a nutrient medium to accumulate the FKI-3368 substance in the nutrient medium and collecting the FKI-3368 substance from the culture.

The FKI-3368 substance producing microbe mentioned above belongs to genus Penicillium and, for example, the strain of Penicillium FKI-3368 isolated by the present inventors is an example which can be most effectively used in the present invention. The mycological characteristics of this strain are as follows.

### I. Morphological Characteristics

This strain grew well on Czapek yeast-extract agar medium, 25% glycerin/nitrate medium, malt extract agar medium, potato dextrose agar medium, etc., and the conidia also adhere well on various agar media.

Microscopic observation of the colonies growing on the Czapek yeast-extract agar medium showed that colorless hyphae have partitions and conidiophores ((50 to 250)×(1.0 to 2.5) μm) erected from aerial mycelia and formed no branches. A penicillus is formed at the apex of the conidiophore. The Penicillus is dimate with 2 to 4 phialides growing closely. The phialide has an ampule-like shape having a size of (6.5 to 7.5)×(2.5 to 3.5) μm. Phialo-type conidia are formed at the apex of the phialide and become a chain-like shape with passage of time. The conidium was spherical to semispherical, grayish brown, having a size of (2.0 to 2.5)×(2.0 to 3.0) μm and a rough surface.

### II. Appearance on Various Culture Media

The results of visual observation after cultured on various culture media at 25° C. for seven days are as in the following Table 1.

TABLE 1

| | Growth state | | | |
|---|---|---|---|---|
| | (diameter of | | | |
| | colony) on | Color of the | Color of rear | |
| Culture | the culture | colony | side of the | Soluble |
| medium | medium | surface | colony | pigment |
| Czapek yeast | Good (50 to | Mignonette | Pale | None |
| extract agar | 55 mm) | Pale yellow | yellowish- | |
| medium | Velvet-like | at | brown | |
| | Wrinkled | peripheral | | |
| | Smooth | | | |
| | peripheral | | | |
| 25% glycerin- | Supressive | Mignonette | Yellow | None |
| nitric agar | (17 to 18 mm) | | | |
| medium | Velvet-like | | | |
| | Smooth | | | |
| | peripheral | | | |
| Malt wort agar | Good | Mignonette | Pale grayish | None |
| medium | (55-57 mm) | White at | yellow | |
| | Velvet-like | peripheral | | |
| | Smooth | | | |
| | peripheral | | | |
| Potato | Good (49 to | Mignonette | Yellow | None |
| dextrose agar | 52 mm) | | | |
| medium | Velvet-like | | | |
| | Smooth | | | |
| | peripheral | | | |

### III. Various Physiological Properties

(1) Optimum growth condition: The most suitable growth condition of this strain is pH 4 to 6, temperature 15.4 to 36.0° C.
(2) Range of growth: The growth range of this strain is pH 2 to 8, temperature 7.6 to 38.0° C.
(3) Aerobic or anaerobic: Aerobic

The results of comparison with known species of microbes based on the above-mentioned morphological characteristics, appearance on various culture media and a physiological properties revealed that this strain belongs to Penicillium sp. On this account, this strain was named as Penicillium sp. FKI-3368 and deposited to the International Patent Organism Depository of independent administrative agency Advanced Industrial Science and Technology (Central 6, Higashi, Tsukuba, Ibaraki) on Sep. 19, 2007 (accession number: FERMP-21369).

As a preferable strain of the present invention, FKI-3368 substance producing microbe has been described but it is easy to mutate its mycological characteristics as is common in the case of microbes, and accordingly, it is a well-known fact that the strain is not constant or rather may be mutated by way of artificial mutation means such as natural or ordinarily performed ultraviolet irradiation, X-ray irradiation, mutation inducing agent, for example, N-methyl-N′-nitro-N-nitrosoguanidine, ethyl methanesulfonate. Not to mention these artificially mutated strains, all the strains, including naturally mutated strains, which belong to Penicillium and have an ability of producing an FKI-3368 substance can be used for the present invention. In addition, strains mutated by cell engineering such as cell fusion or gene manipulation are also included as FKI-3368 substance producing microbes.

An FKI-3368 substance producing microbe belonging to Penicillium is cultured in or on a suitable nutrient medium at first for the production of FKI-3368 substances of the present invention. In the culture of such a strain of the present invention, an ordinary fungal culture method is applied generally. As for the culture media, nutrient media containing a carbon source which the microbe can assimilate, a nitrogen source which the microbe can digest and further inorganic salts as needed are used appropriately.

Glucose, cane sugar, molasses, starch, dextrin, cellulose, glycerin, organic acids may be used singly or in combination as the assimilable carbon source mentioned above. Organic nitrogen source such as such as peptone, meat extract, yeast extract, dried yeast, soybean meal, corn steep liquor, cottonseed meal, casein, soy protein hydrolysate, amino acids and urea, inorganic nitrogen compounds such as nitrates and ammonium salts may be used singly or in combination as the digestible nitrogen source.

In addition, inorganic salts such as sodium salts, potassium salts, calcium salts, magnesium salts and phosphates, heavy metal salts may be added as needed. Furthermore, micronutrients, growth promoters and precursors which promote the growth of the microbe of the present invention and/or the production of FKI-3368 substances may be added as needed, properly in the culture medium.

It is usually preferable to perform culture under aerobic conditions such as shaking culture or aeration stirring culture. Industrially, submerged aeration culture is preferable. The pH of the culture medium is preferably around neutrality. The culture temperature may be in the range of 20 to 37° C., but the temperature is ordinarily maintained to the range of 24 to 30° C., preferably around 27° C. As for the culturing time, the FKI-3368 substance of the present invention is ordinarily produced and accumulated when culturing is performed for 10 to 15 days and therefore, the culturing may be preferably finished when the accumulated FKI-3368 substance reaches the maximum level.

Needless to say, these culture conditions such as the culture composition, pH of the culture medium, culture temperature, stirring rate and aeration rate may appropriately be adjusted and/or selected so that desirable results may be obtained depending on the kind of the strain to use and/or the external conditions. When foaming occurs in liquid culturing, an antifoaming agent such as silicone oil, vegetable oil and a surfactant may be used appropriately. Since the FKI-3368 substance accumulated in the culture is contained in culture filtrate or cultured microbe bodies, it is advantageous to filter the culture filtrate with a filtration adjuvant such as cerite or a high-flow supercell as needed or centrifuge the culture filtrate to separate the culture filtrate and the microbe bodies and concentrate the extract with an organic solvent of the culture filtrate and the microbe bodies and take out the FKI-3368 substance therefrom.

In order to take out the FKI-3368 substance from the culture filtrate, the culture filtrate is extracted with a non-hydrophilic organic solvent such as ethyl acetate, butyl acetate, butanol and chloroform at first, and the extract is vacuum concentrated to obtain a crude FKI-3368 substance. The crude substance can be further subjected to known methods usually used for purification of lipophilic substances such as column chromatography using carriers such as silicagel or alumina to separate and purify the FKI-3368 substance.

In order to take out the FKI-3368 substance from the microbe bodies, they are extracted with a hydrous hydrophilic organic solvent such as hydrous acetone, hydrous methanol or hydrous ethanol, and the obtained extract is vacuum concentrated and the concentrate is extracted with a non-hydrophilic organic solvent such as ethyl acetate, butyl acetate, butanol and chloroform, and the obtained extract may be combined with the extract obtained from the culture liquid and then subjected to separation and purification, or the FKI-3368 substance may be separated and purified by the same method as above.

### C. Use of the FKI-3368 Substance

The novel FKI-3368 substances of the present invention include a compound having antibiotic activity as above and therefore, they can be used as antibiotics, pharmaceutical agents and antibacterial agents.

### EXAMPLES

In the following, the present invention is specifically described by way examples but the present invention is not limited to only these.

### Example 1

100 ml of a nutrient medium (adjusted to pH 6.5) containing yeast extract (0.2%), glucose (2.0%), polypeptone (0.5%), MgSO₄.7H₂O (0.05%), KH₂PO₄ (0.1%) and agar (0.1%) was placed in a 500 ml-Erlenmeyer flask and, after provided with a cotton plug, the flask was autoclaved in the conditions at 121° C. for 20 minutes. After the flask was cooled off sufficiently, spores of Penicillium sp. FKI-3368, which had been grown on an agar medium, were aseptically inoculated and subjected to shaking culture at 27° C. for three days to obtain seed culture liquid.

A culture medium (pH not adjusted) containing 50 g of Italian rice and 0.5 g of seaweed drink powder was placed in a 500 ml-Erlenmeyer flask and 20 sets of these were prepared. After provided with a cotton plug, the flasks were autoclaved in the conditions at 121° C. for 20 minutes. After the flasks were cooled off sufficiently, 2 ml of the seed culture liquid mentioned above per Erlenmeyer flask was aseptically inoculated and cultured at 27° C. for 15 days. 100 ml of 50% ethanol solution was added to each Erlenmeyer flask and the resultant extract was filtrated under reduced pressure to obtain an ethanol extract. After this extract was concentrated under reduced pressure and diluted to 1 liter of an aqueous solution, the same volume of ethyl acetate was added thereto for the purpose of extraction, and the mixture was separated to an aqueous layer and an ethyl acetate layer with a separatory funnel.

After 500 g of anhydrous sodium sulfate was added to the ethyl acetate layer to dehydrate the latter, the ethyl acetate layer was concentrated under reduced pressure to obtain 3.32 g of crude substance I. This was dissolved in a small amount of chloroform and loaded on the top of a silica gel column (MerckArt. 7734; inner diameter: 30 mm, height: 300 mm) filled up with chloroform. After washed with chloroform, the column was eluted with chloroform-methanol (99:1) and the elute was vacuum concentrated to obtain 0.81 g of crude substance II.

This was dissolved in a small amount of hexane and loaded on the top of a silica gel column (MerckArt. 7734; inner diameter: 30 mm, height: 300 mm) filled up with hexane. After washed with hexane, the column was eluted with hexane-ethyl acetate (60:40 and 55:45) and the elute was vacuum concentrated to obtain 0.29 g of crude substance III.

This crude substance III was dissolved in a small amount of ethanol and injected into a high performance liquid chromatography (PEGASIL ODS, 20 mm (diameter)×250 mm, produced by Senshu Scientific Co. Ltd., Japan) and elution was performed with 60% acetonitrile containing 0.05% phosphoric acid as a mobile phase while absorption of 210 nm was detected, and peaks which elute at 25 minutes and 50 minutes at a flow rate of 7 ml/min were collected. These were vacuum concentrated and after acetonitrile was evaporated, the residue was extracted with ethyl acetate. The ethyl acetate layer was separated and dehydrated and then vacuum concentrated to obtain 63.9 mg of yellow powder of undecaprenyl diphosphate synthetase inhibitor FKI-3368-I substance and FKI-3368-II substance and 1.00 mg of yellow powder of FKI-3368-II substance.

### (II) FKI-3368-1 Substance

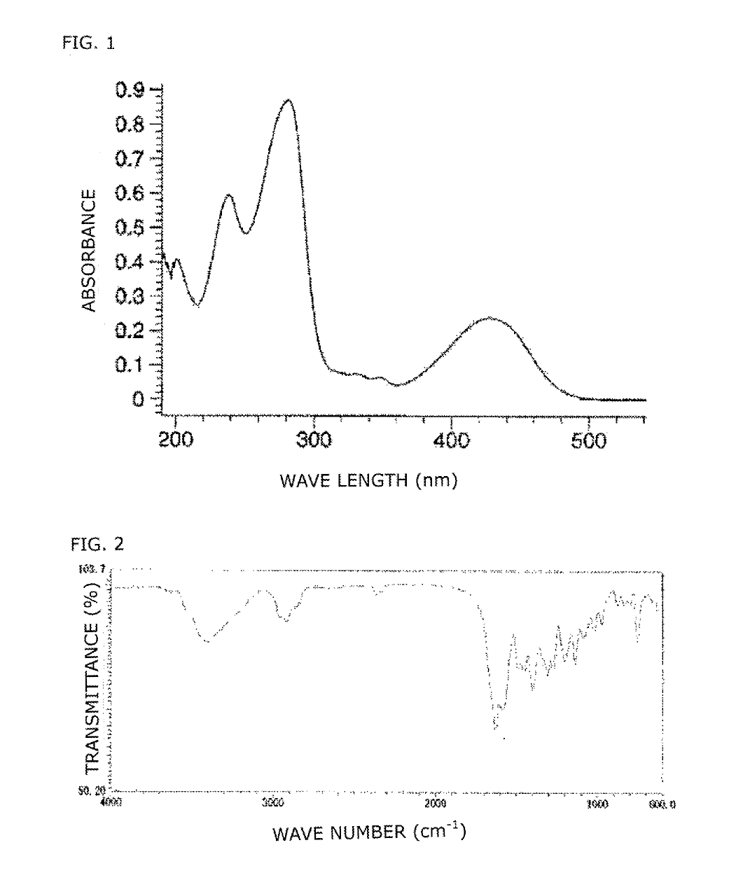

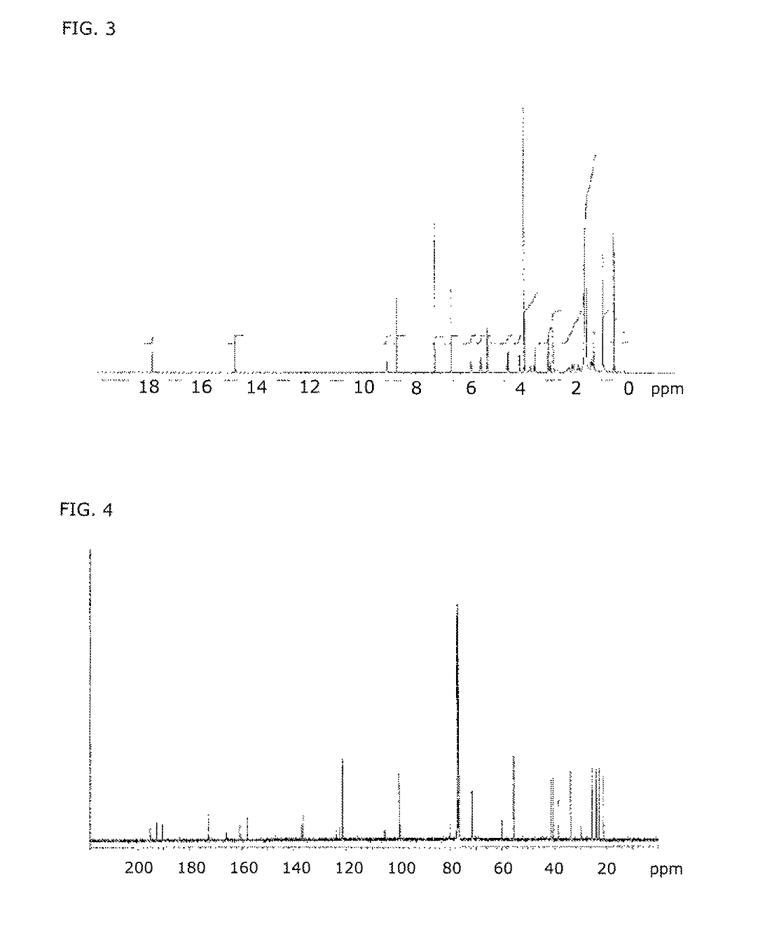

(1) Molecular formula: C₃₀H₃₁NO₁₀ (m/z 564.1854 (M-H) was observed by a high resolution FAB mass spectrum) (calculation value: 565.1948).
(2) Molecular weight: 538 (m/z 561 (M+Na)⁺ was observed in the FAB mass spectrum).
(3) Specific rotation: [α]_{D}²⁴+17° (c=1.0, ethanol).
(4) Ultraviolet absorption spectrum: The ultraviolet absorption spectrum measured in methanol is as shown in FIG. 1, which shows a characteristic absorption maximum respectively at 239 nm (ε=18,700), 281 nm (ε=26,600) and 427 nm (ε=7,870).
(5) Infrared absorption spectrum: The infrared absorption spectrum measured by potassium bromide disk method is as shown in FIG. 2, which shows characteristic absorption bands at λmax(KBR) cm⁻¹: 3401, 2960, 2915, 1627 and 1587.
(6) Solubility in solvents: Soluble in methanol, ethanol, acetonitrile, ethyl acetate, chloroform and dimethylsulfoxide and indissoluble in water.
(7) Basic, acid or neutral: Neutral.
(8) Color and appearance of the substance: Yellow powdery substance.
(9) Proton nuclear magnetic resonance spectrum: The proton nuclear magnetic resonance spectrum (measured in heavy methanol, 600 MHz) obtained by using a nuclear magnetic resonance spectrometer produced by Varian Corporation is as shown in FIG. 3, which shows chemical shifts (ppm) as shown in Table 2.

TABLE 2

| No. | ¹³C | ¹H |
|---|---|---|
| 1 | 190.4s | |
| 2 | 99.56s | |
| 3 | 192.85s | |
| 3-OH | | 17.9s |
| 4 | 40.6t | 2.76d, 2.82d |
| 4a | 71.1s | |
| 4a-OH | | 4.15s |
| 5 | 71.5d | 4.5s |
| 5-OH | | 3.04s |
| 5a | 123.7s | |
| 6 | 137.1s | |
| 6a | 147.1s | |
| 7 | 122.7s | |
| 8 | 160.7s | |
| 9 | 99.9d | 6.65s |
| 10 | 157.9s | |
| 10-OH | | 8.68br.s |
| 10a | 105.4s | |
| 11 | 165.9s | |
| 11-OH | | 14.79br.s |
| 11a | 105.1s | |
| 12 | 195.3s | |
| 12a | 80.6s | |
| 12a-OH | | 5.28br.s |
| 13 | 172.77s | |
| 13-NH₂ | | 5.92br.s, 9.07br.s |
| 14 | 41.1t | 2.93d, 3.44d |
| 15 | 60.1s | |
| 16 | 136.5s | |
| 17 | 121.4d | 5.51m |
| 18 | 22.8t | 2.02m, 2.2m |
| 19 | 33.8t | 1.34m, 1.84m |
| 20 | 38.5s | |
| 21 | 21q | 1.53s |
| 22 | 25.4q | 0.47s |
| 23 | 23.9q | 0.91s |
| 24 | 55.5q | 3.87s |

(10) Carbon nuclear magnetic resonance spectrum: The carbon nuclear magnetic resonance spectrum (measured in heavy methanol, 150 MHz) obtained by using a nuclear magnetic resonance spectrometer produced by Varian Corporation is as shown in FIG. 4, which shows chemical shifts (ppm) as shown in Table 2.

As a result of examining the various physical and chemical properties and spectrum data of the FKI-3368-1 substance in detail, it was determined that the FKI-3368-1 substance has the following chemical structure represented by the formula (II).

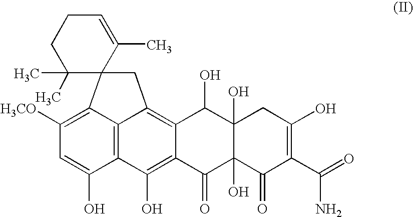

### (III) FKI-3368-2 Substance

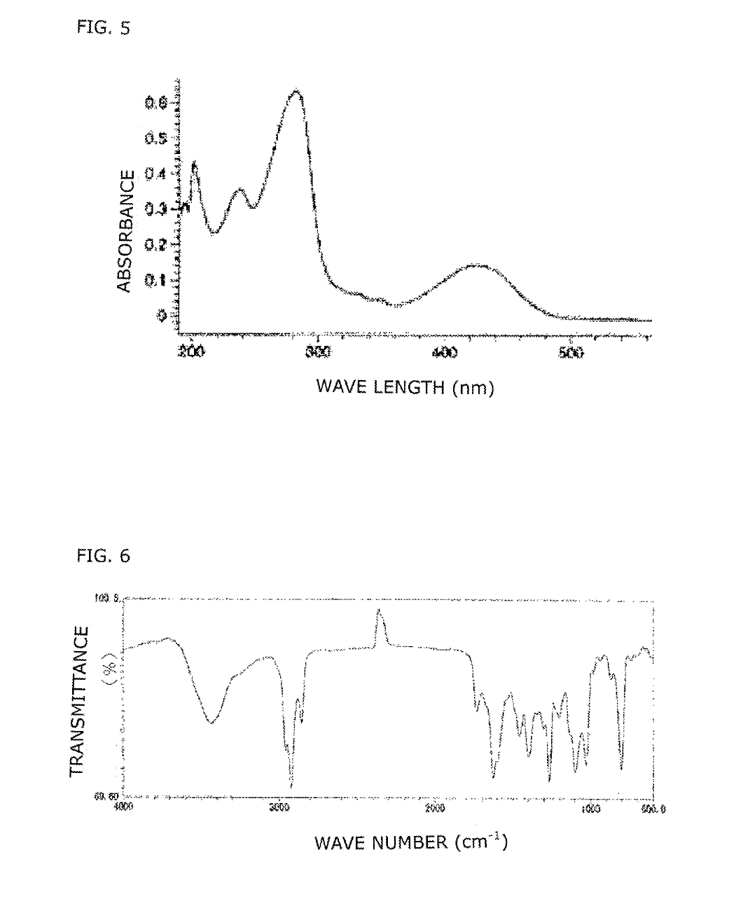

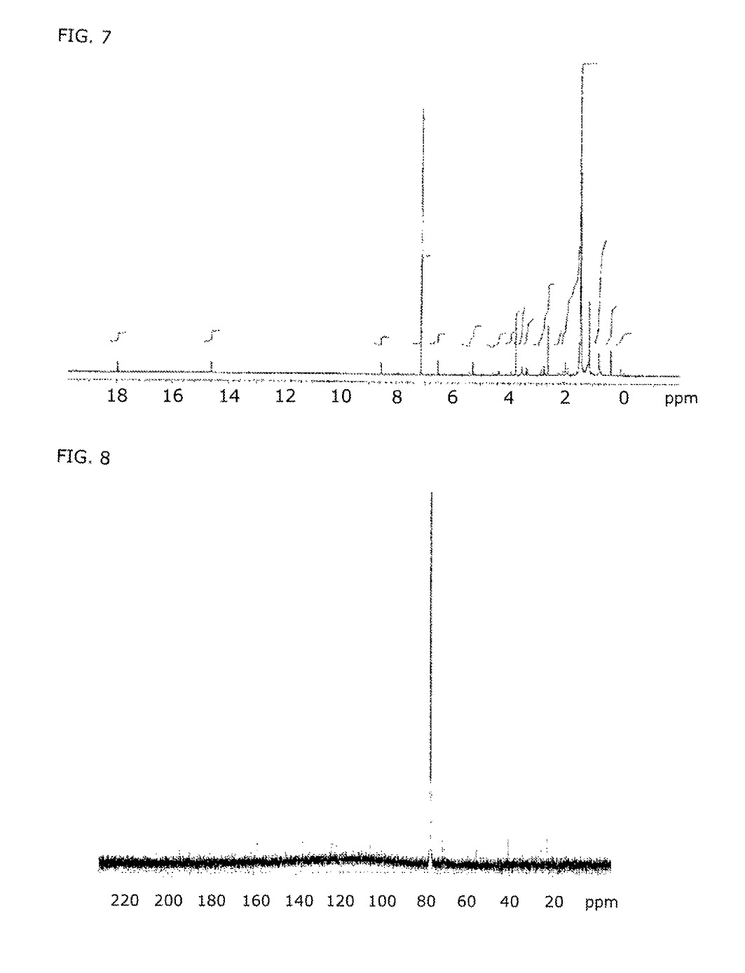

(1) Molecular formula: C₃₁H₃₂NO₁₀ (m/z 563.1900 (M−H)− was observed by a high resolution FAB mass spectrum) (calculation value: 563.1917).
(2) Molecular weight: 552 (m/z 575 (M+Na)⁺ was observed in the FAB mass spectrum).
(3) Specific rotation: [α]_{D}²⁴+13° (c=1.0, ethanol)
(4) Ultraviolet absorption spectrum: The ultraviolet absorption spectrum measured in methanol is as shown in FIG. 5, which shows a characteristic absorption maximum respectively at 238 nm (ε=20,100), 282 nm (ε=35,800) and 423 nm (ε=8,080).
(5) Infrared absorption spectrum: The infrared absorption spectrum measured by potassium bromide disk method is as shown in FIG. 6, which shows characteristic absorption bands at λmax(KBR) cm⁻¹: 3438, 2958, 2925, 1733, 1625 and 1596.
(6) Solubility in solvents: Soluble in methanol, ethanol, acetonitrile, ethyl acetate, chloroform and dimethylsulfoxide and indissoluble in water.
(7) Basic, acid or neutral: Neutral.
(8) Color and appearance of the substance: Yellow powdery substance.
(9) Proton nuclear magnetic resonance spectrum: The proton nuclear magnetic resonance spectrum (measured in heavy methanol, 600 MHz) obtained by using a nuclear magnetic resonance spectrometer produced by Varian Corporation is as shown in FIG. 7, which shows chemical shifts (ppm) as shown in Table 3.

TABLE 3

| No. | ¹³C | ¹H |
|---|---|---|
| 1 | 190.2 | |
| 2 | 110.5 | |
| 3 | 194.4 | |
| 3-OH | | 18.1s |
| 4 | 40.6 | 2.82d, 2.88d |
| 4a | 71.1 | |
| 4a-OH | | |
| 5 | 71.5 | 4.48br.s |
| 5-OH | | |
| 5a | 123.7 | |
| 6 | 137.1 | |
| 6a | 147.1 | |
| 7 | 122.7 | |
| 8 | 160.7 | |
| 9 | 99.9 | 6.65s |
| 10 | 157.9 | |
| 10-OH | | 8.68s |
| 10a | 105.4 | |
| 11 | 165.9 | |
| 11-OH | | 14.73s |
| 11a | 105.1 | |
| 12 | 195.3 | |
| 12a | 80.6 | |
| 12a-OH | | 5.4br.s |
| 13 | 202.2 | |
| 14 | 41.1 | 2.93d, 3.44d |
| 15 | 60.1 | |
| 16 | 136.5 | |
| 17 | 121.4 | 5.51m |
| 18 | 22.8 | 2.02m, 2.2m |
| 19 | 33.8 | 1.34m, 1.84m |
| 20 | 38.5 | |
| 21 | 20.9 | 1.53s |
| 22 | 25.4 | 0.48s |
| 23 | 23.9 | 0.91s |
| 24 | 55.5 | 3.87s |
| 25 | 27.7 | 2.73s |

(10) Carbon nuclear magnetic resonance spectrum: The carbon nuclear magnetic resonance spectrum (measured in heavy methanol, 150 MHz) obtained by using a nuclear magnetic resonance spectrometer produced by Varian Corporation is as shown in FIG. 8, which shows chemical shifts (ppm) as shown in Table 3.

As a result of examining the various physical and chemical properties and spectrum data of the FKI-3368-2 substance in detail, it was determined that the FKI-3368-2 substance has the following chemical structure represented by the formula (III).

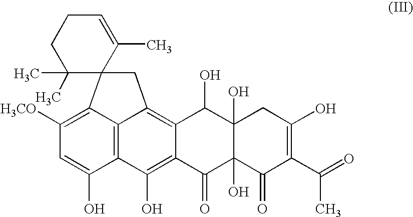

As described heretofore, various physical and chemical properties of the FKI-3368-1 substance and the FKI-3368-2 substance have been described in detail, and since the compounds which agree with such properties have not been reported at all so far and accordingly, it has been concluded that the FKI-3368-1 substance and the FKI-3368-2 substance are novel substances.

### Example 2

Next, biological properties of the FKI-3368-1 substance of the present invention are described below.

(1) Inhibitory effect on the undecaprenyl diphosphate synthase derived from Staphylococcus aureus

The test on the activity of undecaprenyl diphosphate synthase was determined following a method of LI et al. (J. Biomol. Screen., vol. 8, pages 712-715, 2003) with partial changes. The undecaprenyl diphosphate synthase gene derived from Staphylococcus aureus was amplified by PCR and the undecaprenyl diphosphate synthase gene is induced and expressed in E. Coli BL21 (DE3) strain using a promoter derived from T7 bacteriophage while Isopropyl-1-thio-β-D-galactopyranoside was added thereto so that the concentration thereof might be 1 mM, and a cell-free extract of the thus transformed E. Coli was used as the enzyme source.

The transformed E. Coli mentioned above was suspended in a ice-cooled buffer solution A (100 mM Tris-HCl buffer solution (pH 7.5), protease inhibitor cocktail tablet mini) and the microbe bodies were crushed with a French press at a pressure of 1,000 kg/cm². This was centrifuged at 18,800×g at 4° C. for 10 minutes and the obtained supernatant was further centrifuged at 100,000×g, 4° C., for 90 minutes. A cell-free extract was prepared by adding the buffer solution A mentioned above so that the resultant supernatant might have a protein concentration of 10.0 mg/ml.

The measurement of the activity of undecaprenyl diphosphate synthase was conducted as follows: the FKI-3368 substance was added to a buffer solution B (100 mM Tris-HCl buffer solution (pH 7.5), 50 mM potassium chloride, 0.5 mM magnesium chloride, 0.5 μM farnesyl diphosphate, 3.5 μM isopentenyl diphosphate, 50 mM inorganic pyrophosphatase) so that 90 μL in total volume might be respectively put in each well of a 96-well microplate. 10 μL of an enzyme solution (62 μg/ml) was added thereto so that the total volume might amount to 100 μL and after reaction was performed at 37° C. for 20 minutes, 10 μL of 0.5M EDTA was added thereto to terminate the reaction. In order to quantify the released phosphate, 100 μL of a solution C (0.5M Tris-HCl buffer solution (pH 8.8), 1.5 mM inosine, 50 μM Amplex™ Red, 0.02 U nucleoside phosphorylase, 1 U peroxidase, 0.4 U xanthine oxidase) was added to each well and after reaction was performed at room temperature for 40 minutes, fluorescence intensity was measured under the conditions of an excitation wavelength at 544 nm and a measurement wavelength at 590 nm.

The addition amount of farnesyl 2-phosphate and the intensity of fluorescence (F.I.) measured in a reaction liquid without the addition thereof were applied to the following expression and the inhibition % was calculated:

Inhibitory ratio={1−(F.I.(Sample FPP+)−F.I.(Sample FPP−))/(F.I.(Solvent FPP+)−F.I.(Solvent FPP−))}×100

From the calculation results, the concentration at which the undecaprenyl diphosphate synthase activity was inhibited to 50% (IC₅₀) was 4.0 μM for the FKI-3368-1 substance and 10.0 μM for the FKI-3368-2 substance. As described in the above in detail, the FKI-3368 substances of the present invention are expected to be useful as antibacterial agents since they exhibit an inhibitory activity on undecaprenyl diphosphate synthetase.

### Example 3

The measurement of the minimal inhibitory concentration (MIC) for various microbes of the FKI-3368-I substance of the present invention was performed by the following method. An ethanol solution of the FKI-3368-I substance was subjected to 2-fold dilution from the concentration of 1,000 μg/ml to prepare 14 stage diluted solutions (1,000 μg/ml, 500 μg/ml, 250 μg/ml, 125 μg/ml, 62.5 μg/ml, 31.3 μg/ml, 15.6 μg/ml, 7.8 μg/ml, 3.9 μg/ml, 2 μg/ml, 1 μg/ml, 0.5 μg/ml, 0.24 μg/ml and 0.12 μg/ml). After 1 ml each of the respective diluted solutions was placed on a round shape dish (90 mm (diameter)×20 mm, produced by IWAKI Co., Ltd.), 9 ml of a culture medium-N for sensitivity disc (for bacteria; produced by Nissui Pharmaceutical Co., Ltd.) or Sabouraud glucose agar (for yeast or mold; produced by Nihon Pharmaceutical Co., Ltd.) was added thereto to form plate media. The test microbes had been incubated in advance in Muller Hinton culture media (for bacteria; produced by Nissui Pharmaceutical Co., Ltd.) or potato dextrose agar media (for yeast or mold; produced by Nissui Pharmaceutical Co., Ltd.). On the very date when the test was performed, the resultant microbe suspensions were diluted with an Muller Hinton culture medium or a potato dextrose culture medium and a platinum loop thereof was streak-smeared on the plate media. These streak-smeared plate media was incubated at 37° C. for 18 hours (for bacteria) or at 27° C. for 40 hours (for yeast and mold) and MIC was determined.

The results are shown in Table 4.

TABLE 4

| Name of microbes | Strain | MIC (μg/ml) |
|---|---|---|
| Staphylococcus aureus | ATCC 6538P | 0.78 |
| MRSA | K24 | 0.78 |
| Bacillus subtilis | ATCC 6633 | 0.4 |
| Micrococcus luteus | ATCC 9341 | 1.56 |
| Mycobaderium smegmatis | ATCC 607 | >100 |
| Escherichia coli | NIHJ | >100 |
| Klebsiella pneumoniae | ATCC 10031 | >100 |
| Pseudomonas aeruginosa | IFO 3080 | >100 |
| Serratia marcescens | IAM 1021 | >100 |
| Candida albicans | ATCC 64550 | 6.25 |
| Saccharomyces cerevisiae | ATCC 9763 | 12.5 |
| Mucor racemosus | IFO 4581 | 25 |
| Aspergillus niger | ATCC 9642 | 12.5 |
| Penicillium chrysogenam | IAM 12842 | 6.25 |

It has been made definite from these that the FKI-3368-I substance of the present invention has excellent antimicrobial activities.

As described above, substances having an inhibitory activity on the undecaprenyl diphosphate synthetase were obtained by culturing a microbe belonging to Penicillium and having ability to produce FKI-3368 substances in/on a culture medium and collecting the FKI-3368 substances from the culture. It can be expected that the said substances have an effect as an antibacterial agent for intractable bacterial infections by multiple drug resistant bacteria.

What is claimed is:

1. A compound represented by Formula (I): 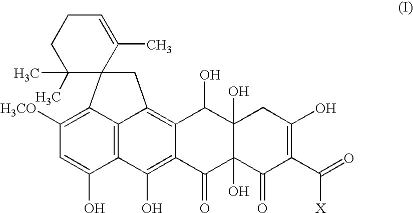
wherein X is a hydroxyl group, an alkyl group having 1 to 3 carbon atoms which may be substituted with a hydroxyl group, a halogen atom, an alkoxy group or an amino group; or an amino group which may be substituted with a hydroxyl group, a halogen atom, an alkoxy group or an amino group (hereinbelow referred to as FKI-3368 substance(s)) or a pharmaceutically acceptable salt or derivative thereof.

2. The compound according to claim 1 having an antibiotic activity.

3. The compound according to claim 1 having an undecaprenyl diphosphate synthase inhibitory activity.

4. The compound according to claim 1, wherein X is a methyl group.

5. The compound according to claim 1 wherein X is an amino group.

6. A production process of the compound according to claim 1 which comprises culturing a microbe belonging to Penicillium and having ability of producing the FKI-3368 substance in or on a nutrient medium to accumulate the FKI-3368 substance in the nutrient medium and collecting the FKI-3368 substance from the culture.

7. A microbe belonging to Penicillium and having ability of producing an FKI-3368 substance.

8. A microbe belonging to Penicillium and having ability of producing an FKI-3368 substance according to claim 7 having an antibiotic activity.

9. A microbe belonging to Penicillium and having ability of producing an FKI-3368 substance according to claim 7 having an undecaprenyl diphosphate synthase inhibitory activity.

10. A pharmaceutical composition containing a compound according to claim 1.

11. An antibacterial agent containing a compound according to claim 1.
